(19) United States
(12) Patent Application Publication
(10) Pub. No.: US 2019/0290332 A1
(43) Pub. Date: Sep. 26, 2019

(54) BONE FIXATION STRUCTURE

(71) Applicant: YU-SAN BIOMEDICAL LTD., Taipei (TW)

(72) Inventors: Fon-Yih TSUANG, TAIPEI (TW); Chang-Jung CHIANG, TAIPEI (TW); Chia-Hsien CHEN, TAIPEI (TW)

(21) Appl. No.: 16/359,291

(22) Filed: Mar. 20, 2019

(30) Foreign Application Priority Data
Mar. 21, 2018 (TW) 107203644

(51) Int. Cl. A61B 17/70; A61B 17/86

(52) U.S. Cl. CPC A61B 17/7035; A61B 17/685; A61B 17/8605; A61B 17/7085

(57) ABSTRACT

A bone screw has a screw body with a spherical head and a screw rod and a positioning sleeve with a connecting rod fixing seat and a calibration part. An annular cutting slot is defined between the connecting rod fixing seat and the calibration part. At least two pairs of connecting rod calibration openings are formed on a sidewall of the calibration part. An axial cutting slot is formed on a sidewall of an adjacent region of the connecting rod calibration openings. The spherical head is connected to a top portion of the screw rod. The screw body is pivoted to a bottom portion of the positioning sleeve. The spherical head is sleevingly arranged on a spherical pit of the connecting rod fixing seat. The calibration part can be split into two pieces and separated from the connecting rod fixing seat. The calibration part is removable from the positioning sleeve.

### FIELD OF THE INVENTION

The present invention relates to a bone fixation structure, and more particularly, to a bone fixation structure applicable to spines.

### BACKGROUND OF THE INVENTION

At present, as humans live longer, our body is more prone to symptoms, for example, nerve compression in the spine. When spinal compression occurs, minimally invasive lumbar spine surgery is often used to fix multiple bone nails on the vertebrae of a particular segment to prevent the vertebrae of that segment from being displaced. This inhibits movement of the vertebrae in that segment, such that, for example, the patient can no longer bend down, and as a result of this, the vertebrae of the segment no longer put pressure on the nerves.

During a minimally invasive lumbar spine surgery, the patient typically lies on his stomach (as shown in FIG. 1), and an incision 80 is formed in the skin surface 8 to implant a plurality of bone nails 1 on vertebrae 8a, 8b, 8c, and the upper portions 10 of the bone nails 1 protrude from the incision 80. Then, a connecting rod (not shown) is inserted into the incision 80 for threading all of the lower portions 11 of the bone nails 1 together. Thereafter, the upper portions 10 of the bone nails 1 are removed and the wound (i.e., the incision 80) is sutured.

However, since the upper portions 10 of the bone nails are cylindrical in shape, they have to be inserted into the incision 80 almost perpendicular to the surface of the bed on which the patient is lying to prevent the upper portions 10 of the adjacent bone nails 1 from interfering with each other. Therefore, the incision 80 is required to have a certain size (e.g. with a width of at least 5 cm). This makes it difficult to minimize the area occupied by the incision 80 on the skin surface 8 for achieving a minimally invasive surgery. The bone nails 1 can be implanted obliquely into the incision 80, but the angle of inclination is still limited by the interference of the cylindrical upper portions 10 of the adjacent bone nails 1. As a result, the size of the incision 80 or the wound cannot be effectively minimized.

In addition, the inventors of the present invention have previously applied for a patent in the related technical field, and it is proposed hereby, which can refer to the U.S. Pat. No. 9,615,855B2 and the Taiwan Patent No. TWI562754.

Therefore, there is a need for a solution that addresses the aforementioned shortcomings in the prior art.

### SUMMARY OF THE INVENTION

In view of the aforementioned shortcomings of the prior art, the present invention provides a bone fixation structure, which may include: a fastening body; a positioning member including a first end and a second end opposite to each other, the first end being provided at an end of the fastening body; and a holding piece provided on the second end of the positioning member, and a side of the holding piece including an inclined surface.

In the bone fixation structure above, the fastening body is in the shape of a screw.

In the bone fixation structure above, the positioning member and the fastening body are pivotally connected to each other.

In the bone fixation structure above, the positioning member includes a space. For example, the holding piece includes a slot in communication with the space.

In the bone fixation structure above, the holding piece resembles the shape of a spoon or a ladle.

In the bone fixation structure above, a top end of the holding piece is closed or open.

The bone fixation structure above further includes a connecting piece penetratingly disposed on the peripheral surface of the positioning member. The bone fixation structure above further includes a fastening piece for fastening the connecting piece on the positioning member. For example, the fastening piece is screwed inside the positioning member and presses against the connecting piece.

In view of the above, the bone fixation structure proposed by the present invention exploits, in particular, the inclined surface on the holding piece, such that during a spinal fixation surgery, one holding piece can fit snuggly with the inclined surface of an adjacent holding piece. As a result, these holding pieces can be fitted tightly next to each other to reduce distance them and thus the area occupied by the holding pieces on the skin surface. Therefore, compared to the prior art, the bone fixation structure proposed by the present invention significantly reduces the width of the incision on the surgical site to achieve minimization of the extent of the wound.

### BRIEF DESCRIPTION OF THE DRAWINGS

FIG. 1 is a schematic side view illustrating the application of conventional bone nails.

FIG. 2A is a schematic front view of a pre-assembly of a bone fixation structure in accordance with the present invention.

FIG. 2A′ is another embodiment of a schematic front view of a pre-assembly of a bone fixation structure in accordance with the present invention.

FIG. 2B is a schematic rear view of a pre-assembly of the bone fixation structure in accordance with the present invention.

FIG. 2C is a schematic side view of the bone fixation structure in accordance with the present invention.

FIG. 2D is a schematic diagram of a fixation assembly of the bone fixation structure in accordance with the present invention.

FIGS. 3A to 3E are schematic side view illustrating the processes of applying the bone fixation structure in accordance with the present invention.

FIG. 4 are schematic diagram illustrating three possible forms the holding piece of the bone fixation structure in accordance with the present invention may assume.

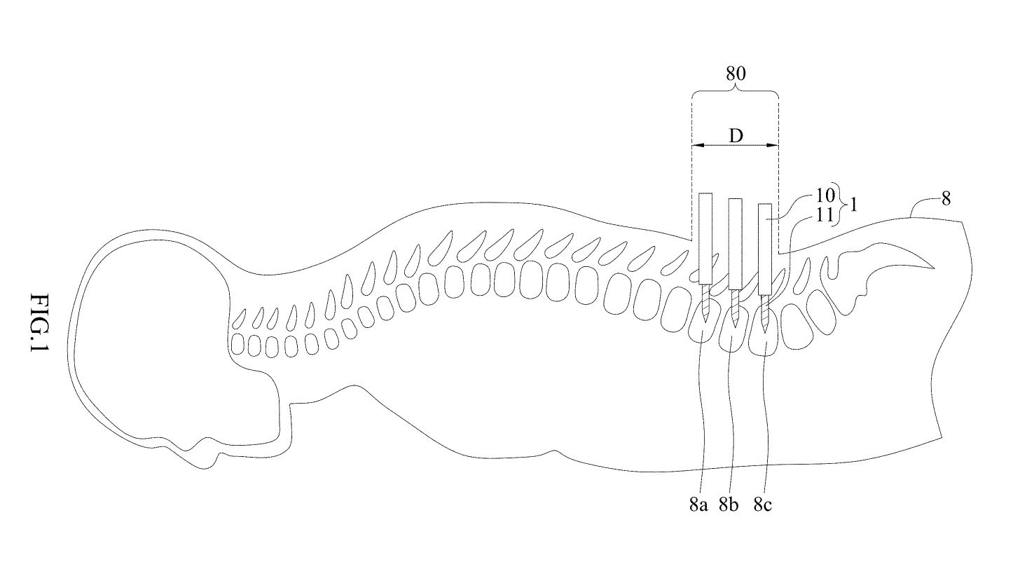

### DETAILED DESCRIPTION OF THE PREFERRED EMBODIMENTS

Aspects of the present invention are described by the following specific embodiments. Other advantages and effects of the present invention can be readily understood by one of ordinary skill in the art upon reading the disclosure of this specification.

It should be noted that the structures, ratios, sizes shown in the drawings appended to this specification are to be construed in conjunction with the disclosure of this specification in order to facilitate understanding of those skilled in the art. They are not meant, in any ways, to limit the implementations of the present invention, and therefore have no substantial technical meaning. Any modifications, changes or adjustments to the structures, ratio relationships or sizes, should be regarded as falling within the range covered by the technical contents disclosed herein to the extent that they do not affect the effects created and the objectives achieved by the present invention. Meanwhile, terms, such as “in”, “top”, “bottom”, “above”, “below”, “one”, “a”, “an”, and the like, are for illustrative purposes only, and are not meant to limit the range implementable by the present invention. Any changes or adjustments made to their relative relationships are also to be regarded as within the range implementable by the present invention to the extent that they do not modify the substantial technical contents of the present invention.

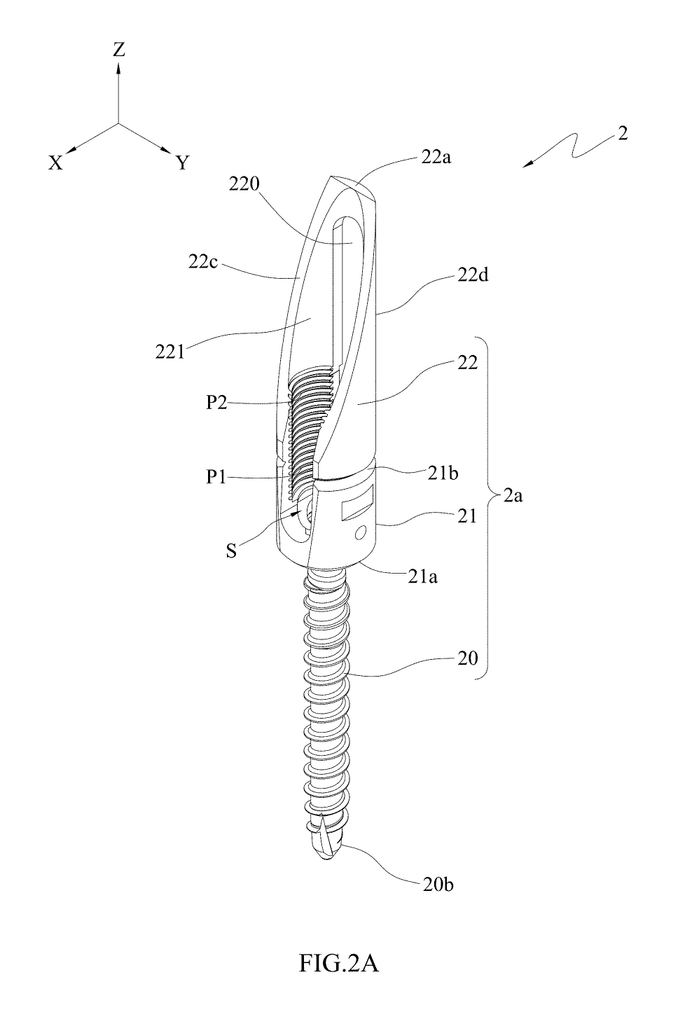

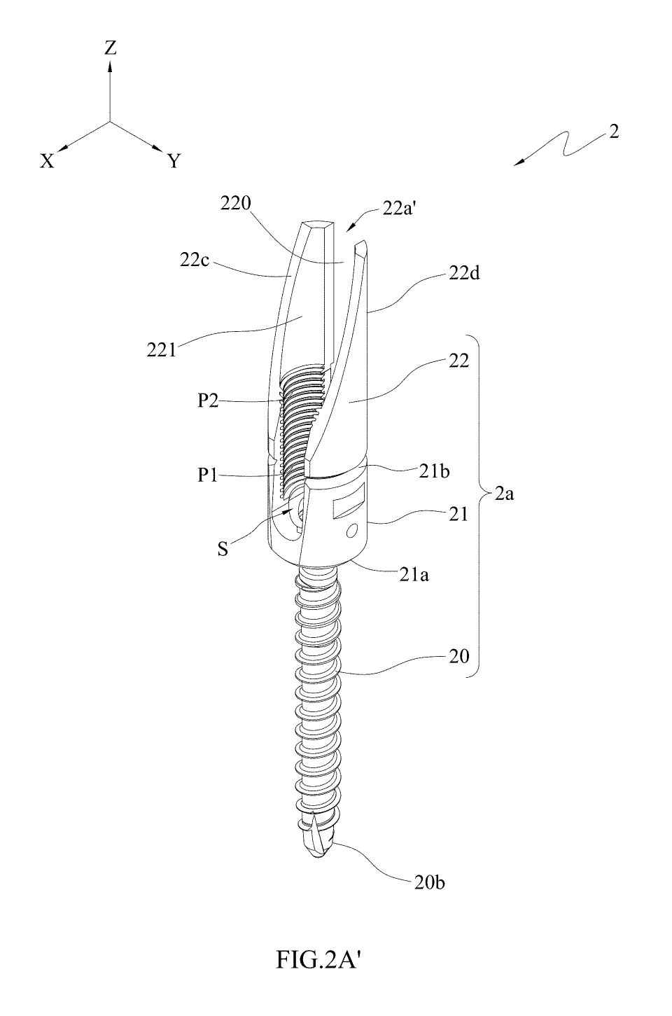

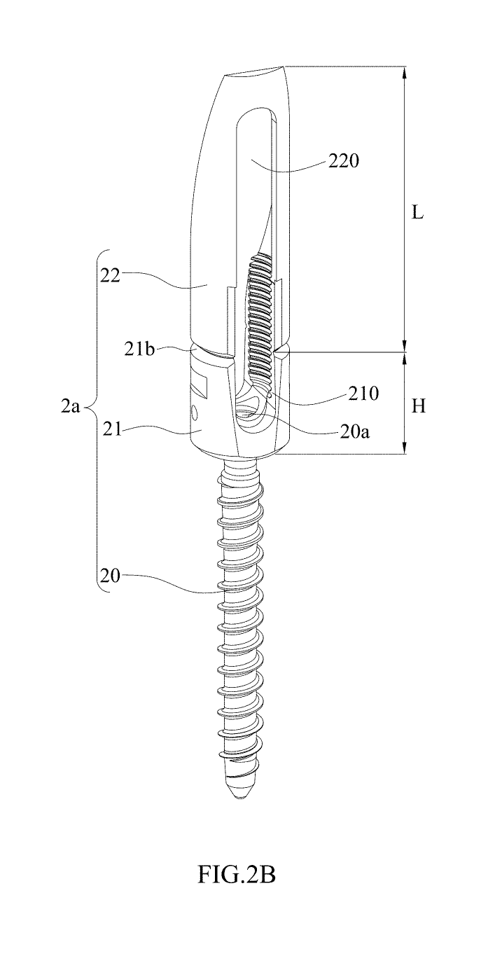

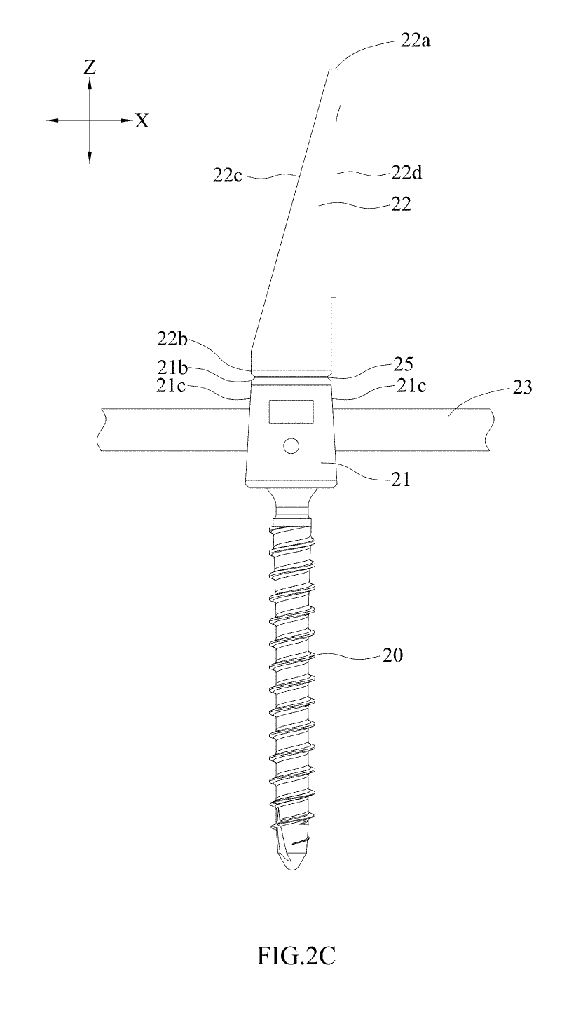

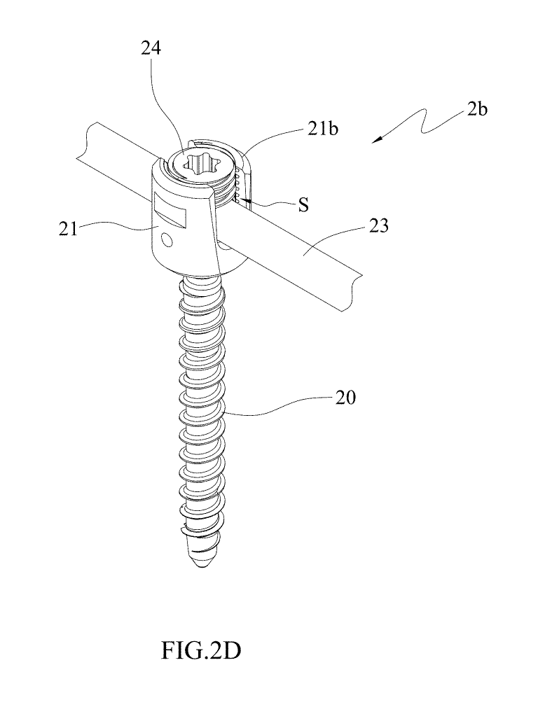

FIG. 2A is a schematic diagram depicting a bone fixation structure 2 in accordance with the present invention. In this embodiment, the bone fixation structure 2 is applied to spines.

As shown in FIGS. 2A and 2B, the bone fixation structure 2 includes: a fastening body 20, a positioning member 21 and a holding piece 22. The fastening body 20, the positioning member 21 and the holding piece 22 are used as a pre-assembly 2a.

The fastening body 20 is in the shape of a metal screw, such as that of a pointed screw, having a head portion 20a and a tip portion 20b. It can be appreciated that the fastening body 20 may assume any forms appropriate for the particular requirements, and is not limited to that just described.

The positioning member 21 includes a first end 21a and a second end 21b opposite to each other. The positioning member 21 is joined with the head portion 20a of the fastening body 20 via its first end 21a.

In this embodiment, the positioning member 21 is an alloy material made substantially into the shape of a cylinder or a hat. The positioning member 21 includes a space S, and the spherical head portion 20a of the fastening body 20 is pivotally connected at the bottom of the space S, such that the positioning member 21 can rotate relative to the fastening body 20 (or the fastening body 20 can rotate relative to the positioning member 21).

Furthermore, threads P1 are provided on the inner surface of the space S. A notch 210 is formed on the side faces of the space S in communication with the second end 21b, such that the notch 210 is in laterally communication with two side faces of the positioning member 21 (e.g. along the X-axis shown by the coordinates in FIG. 2A). For example, the front or back view of the positioning member 21 may assume an “U”-like shape.

The holding piece 22 is an alloy material integrally formed on the second end 21b of the positioning member 21 with a shape roughly resembles a spoon or a ladle.

In this embodiment, the holding piece 22 is configured in such a way that it starts with a hollow cylinder at one end and tapers into a thin piece at the other end. When viewed from the top, the diameter of the hollow circle in the cylinder ranges from 10 mm to 16 mm depending on the various tools used, which have different force requirements. For example, the cylindrical peripheral surface of the holding piece 22 has two opposite sides. One side includes an inclined surface 22c formed by cutting diagonally from the top end of the holding piece 22 to the bottom end situated near the second end 21b of the positioning member 21. The other side includes a curved surface 22d. A recess 221 is formed on the side with the inclined surface 22c, such that the holding piece 22 resembles the shape of a spoon or a ladle.

Moreover, the length L of the holding piece 22 is greater than the length H of the positioning member 21. The length L is in the range of 16 mm to 200 mm, and the length H is in the range of 5 mm to 70 mm.

Furthermore, threads P2 are also provided at the bottom of the recess 221. A slot 220 (as shown in FIG. 2B) is formed on the recess 221 in communication with the space S (i.e. the notch 210), such that the recess 221 and the slot 220 are in lateral communication (e.g. along the X-axis of the coordinates shown in FIG. 2A) with the peripheral surface of the holding piece 22.

Moreover, the top end 22a of the holding piece 22 is closed, but in other embodiments, the top end 22a′ of the holding piece 22 may be open, such as that shown in FIG. 2A′.

In another embodiment, as shown in FIG. 2C, the bone fixation structure 2 further includes a connecting piece 23, which is penetratingly disposed on the peripheral surface 21c of the positioning member 21. For example, the connecting piece 23 is a straight or curved rod made of metal, which can penetrates laterally (e.g. along the X-axis of the coordinates shown in FIG. 2A or 2C) through the positioning member 21 or the holding piece 22 via the notch 210 or the slot 220, respectively, such that it can be shifted in the recess 221 and the space S along a path formed by the slot 220 and the notch 210 (e.g. along the Z-axis shown in FIG. 2A or 2C). In addition, as shown in FIG. 2D, the bone fixation structure 2 further includes a fastening piece 24 for fastening the connecting piece 23 on the positioning member 21. For example, the fastening piece 24 is a metal screw, which can be moved from the threads P2 of the recess 221 to the threads P1 in the space S of the positioning member 21 to fasten the connecting piece 23 in place within the space S.

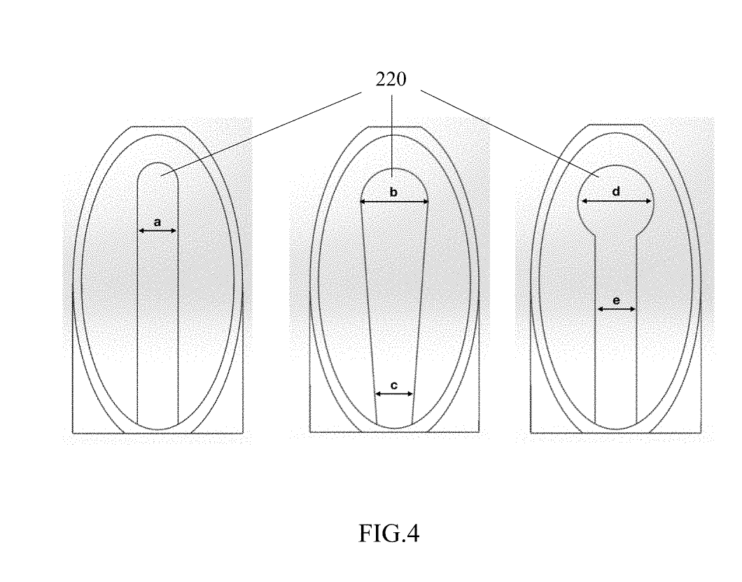

Furthermore, referring to FIG. 4, the slot 220 of the holding piece 22 may come in at least three different forms, including a narrow elongated slot, a fan-shaped slot and a slot having a combination of a circular portion and a narrow elongated portion, wherein the width a of the narrow elongated slot is between 5.5 mm to 7.5 mm; the fan-shaped slot includes a wide portion b between 7.0 mm to 9.5 mm and a narrow portion c between 5.5 mm to 7.5 mm; and the slot having the combination of a circular portion and the narrow elongated portion includes a diameter d of 7.0 mm to 9.5 mm for the circular portion and a width e of 5.5 mm to 7.5 mm for the narrow elongated portion.

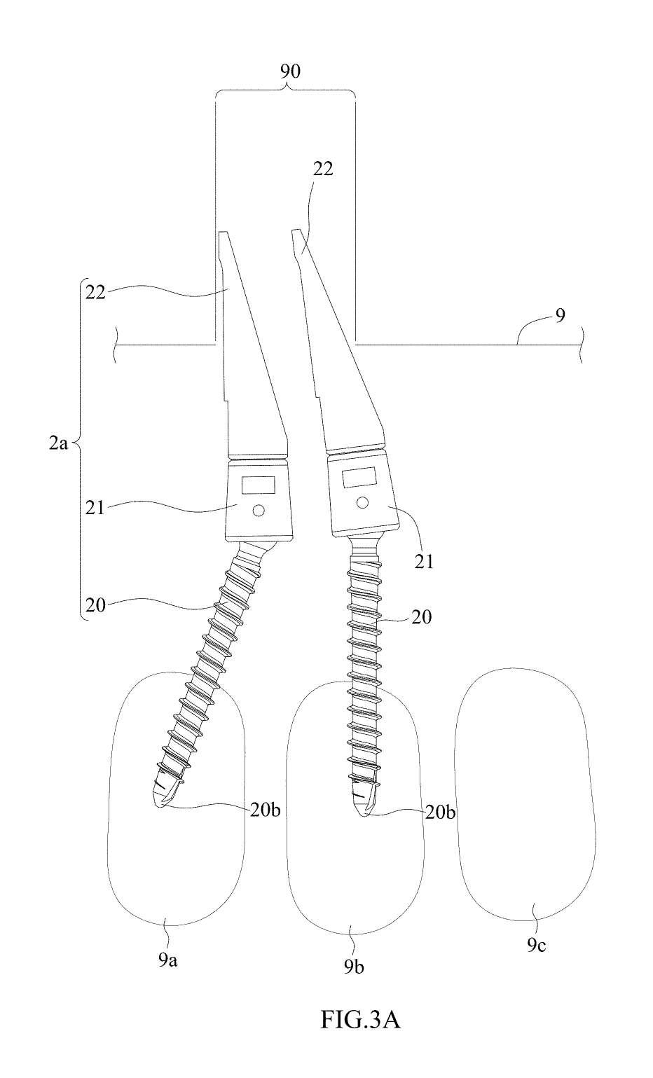

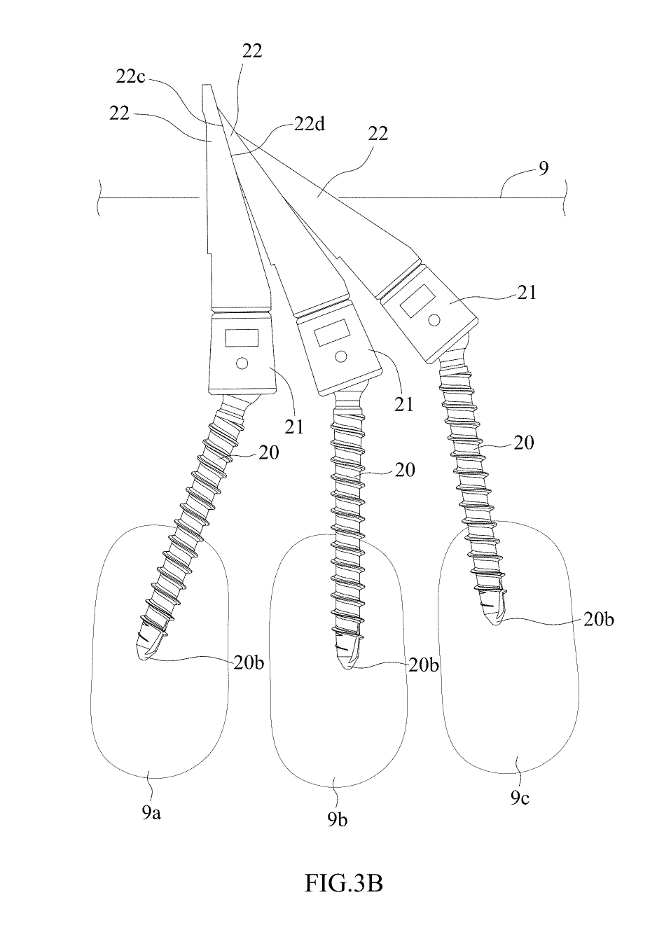

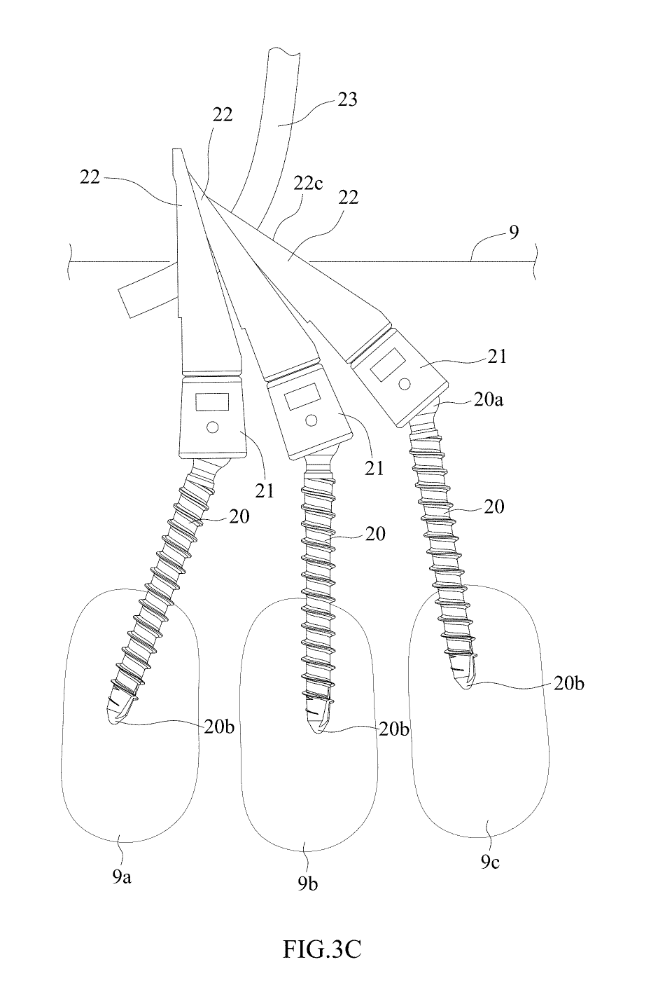

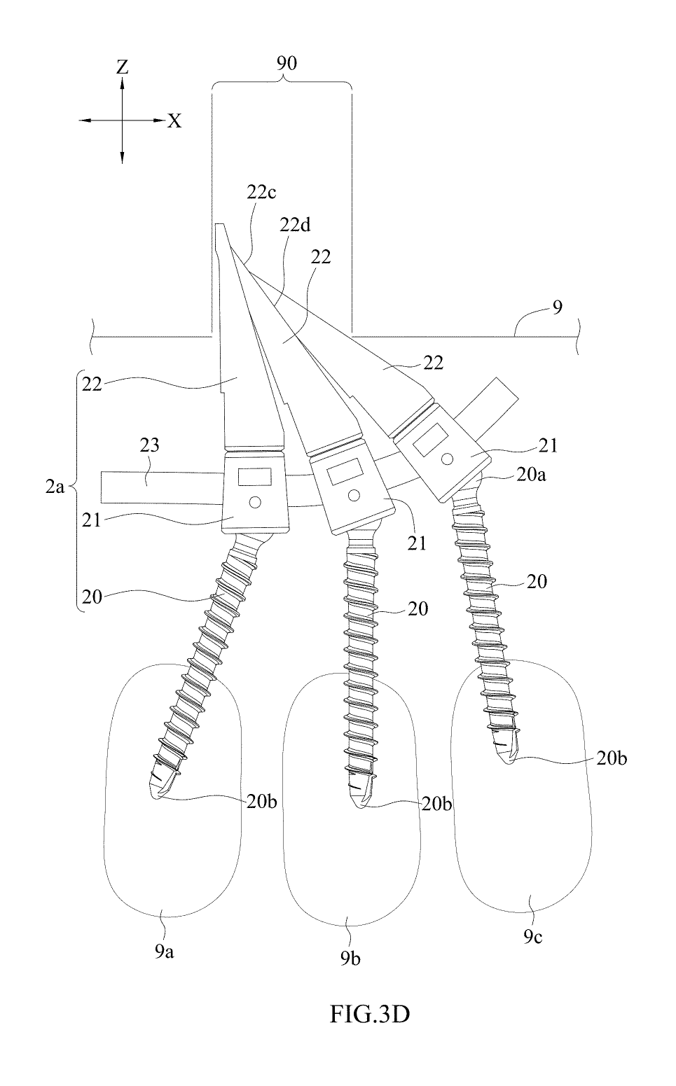

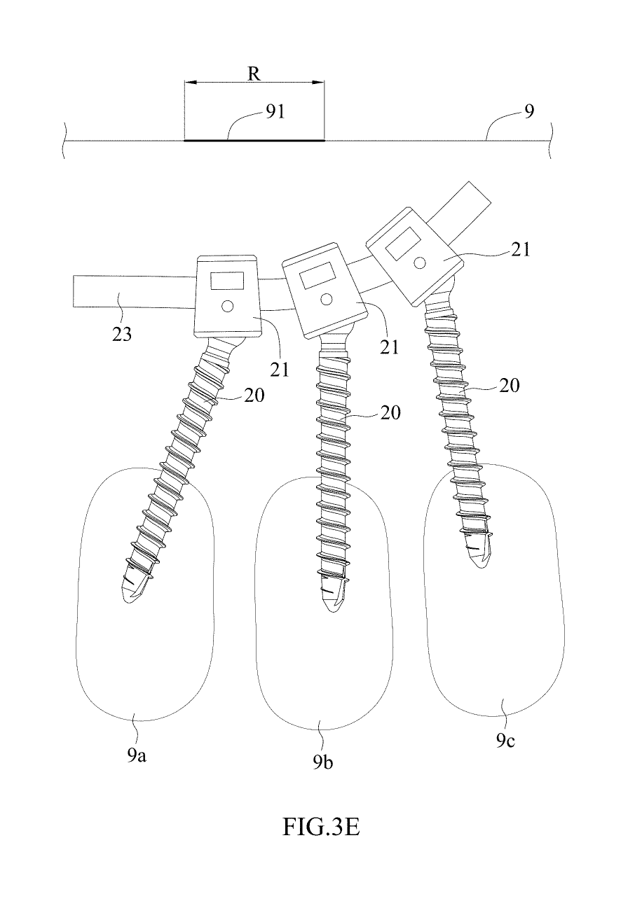

When a spinal fixation surgery is performed (manually or robotically), as shown in FIG. 3A, an incision 90 is created on a skin surface 9 at a location to be fixated (i.e. vertebrates 9a, 9b, 9c), so that the user (i.e. a surgeon) can see the vertebrates 9a, 9b, 9c. Then, the fastening bodies 20 of at least two pre-assembly 2a are implanted into the corresponding vertebrates 9a, 9b, 9c. The fastening bodies 20 along with the positioning members 21 can enter the incision 90 at an angle relative to the skin surface 9 and then be implanted into the corresponding vertebrates 9a, 9b, 9c. As such, it is not necessary for the size of the incision 90 to match a projected area of the vertebrates 9a, 9b, 9c to be fixated. In other words, the incision 90 can be kept relatively small.

As shown in FIG. 3B, since the positioning member 21 can rotate with respect to the fastening body 20, and the positioning member 21 is fixed to the holding piece 22 (e.g. they are formed integrally together), after all the fastening bodies 20 (three pre-assemblies 2a shown in FIG. 3B) are implanted into the appropriate locations (e.g. drilled into the vertebrates 9a, 9b, 9c), the holding pieces 22 can be rotated in such a way that the curved surface 22d of one holding piece 22 fits snuggly with the inclined surface 22c of an adjacent holding piece 22. As a result, these holding pieces 22 can be fitted tightly next to each other. Preferably, a portion of the curved surface 22d (or the top end 22a) of one holding piece 22 is received in the recess 221 of another holding piece 22.

Next, as shown in FIGS. 3C and 3D, a rod-shaped and curved connecting piece 23 is inserted (almost perpendicular to the inclined surfaces 22c) into the slots 220 of the holding pieces 22 and moved downwards along the slots 220 until a portion of the connecting piece 23 has penetrated through the notches 210. Then, the connecting piece 23 is rotated gradually to be embedded completely through the incision 90. At this point, the connecting piece 23 is adjusted laterally (e.g. along the X-axis shown in FIG. 2A or 3D) to an appropriate position, so that the connecting piece 23 is laid substantially flat (e.g. along the X-axis shown in FIG. 3D) in the space S of the positioning members 21 against the head portions 20a of the fastening bodies 20, thereby threading all of the positioning members 21 together.

Subsequently, as shown in FIG. 3D, a plurality of fastening pieces 24 are passed into the incision 90 and engaged in the respective positioning members 21 through the threads in order to exert downward pressure on the connecting piece 23 and securely fasten it in place.

Finally, as shown in FIG. 3E, the holding pieces 22 are removed or severed to suture the wound (i.e. the incision 90) with stitches 91, for example. This completes the spinal fixation surgery. Meanwhile, the fastening bodies 20, the positioning members 21, the connecting piece 23, and the fastening piece 24 form a fixation assembly 2b (as shown in the state in FIG. 2D) for preventing the vertebrates 9a, 9b, 9c from displacements, i.e. the patient can no longer move the vertebrates 9a, 9b, 9c (e.g. he can no longer bend over).

Therefore, with the design of the inclined surfaces 22c of the holding pieces 22 in the bone fixation structure 2 proposed by the present invention, during a spinal fixation surgery, the curved surface 22d of one holding piece 22 can fit snuggly with the inclined surface 22c of an adjacent holding piece 22. This allows the holding pieces 22 to be arranged tightly with minimum intervals between them, which subsequently reduces the area to be occupied by these holding pieces 22 on the skin surface (and thus, the size of the incision 90). As a result, compared to the prior art, a relatively smaller incision 90 can be formed on the surgical site (with a maximum width R of about 3 cm as shown in FIG. 3E), thereby achieving a minimally invasive surgery.

Moreover, a cut 25 can be created between the positioning member 21 and the holding piece 22, for example, between the second end 21b of the positioning member 21 and a bottom end 22b of the holding piece 22 in FIG. 2C, to facilitate the removal or severing of the holding piece 22. Furthermore, the cut 25 forms an angle that is in the range of 40 and 50 degrees. The thickness of the cut 25 from the outer periphery side of the positioning member 21 or the holding piece 22 to the inner wall side (or from the central portion of the cut 25 to the inner wall side) is preferably in the range of 0.1 to 0.15 mm. With such thicknesses and angles, it is easy to break off the holding piece 22 from the positioning member 21 without leaving many raw edges behind.

In addition, during the operation, if the surgeon finds the view through the incision 90 is not clear enough, a holding piece 22 with an open top end 22a′ (shown in FIG. 2A′) can be used for a wider surgical view. On the other hand, a cut (not shown) can be provided on a closed top end 22a of a holding piece 22, and depending on the need at the time of surgery, the closed top end 22a can be broken off to become an open top end 22′ for providing a wider surgical view and for facilitating the insertion of the connecting piece 23 in the incision 90. Also, the cut at the closed top end 22a of the holding piece 22 can have a shape with an angle that is between 40 to 50 degrees. The thickness of the cut 25 from the center portion of the side of the cut 25 to the inner wall side is preferably in the range of 0.1 to 0.15 mm. With such thicknesses and angles, it is easy to break off the closed top end 22a from the holding piece 22 without leaving many raw edges behind.

The above embodiments are used only to illustrate the principles of the present invention and its effect, rather than to limit the present invention. The above embodiments can be modified by one of ordinary skill in the art without departing from the spirit and scope of the present invention. Therefore, the scope claimed of the present invention should be defined by the following claims.

1. A bone fixation structure comprising:
a fastening body;
a positioning member including a first end and a second end opposite to each other, the first end being provided at an end of the fastening body, and the height of the positioning member being between 16 mm to 200 mm; and
a holding piece provided on the second end of the positioning member, a side of the holding piece including an inclined surface, and the height of the holding piece being between 5 to 70 mm, wherein the holding piece is a hollow cylinder with a diameter between 10 mm to 16 mm.

2. The bone fixation structure of claim 1, wherein the fastening body is in the shape of a screw.

3. The bone fixation structure of claim 1, wherein the positioning member and the fastening body are pivotally connected to each other.

4. The bone fixation structure of claim 1, wherein the positioning member includes a space.

5. The bone fixation structure of claim 4, wherein the holding piece includes a slot in communication with the space.

6. The bone fixation structure of claim 5, wherein the holding piece resembles the shape of a spoon or a ladle.

7. The bone fixation structure of claim 6, wherein a cut is provided between the holding piece and the positioning member, the cut forms an angle in the range of 40 degrees to 50 degrees, and the thickness between a central portion of the cut and an inner wall of the holding piece is between 0.1 mm and 0.15 mm.

8. The bone fixation structure of claim 7, wherein a top end of the holding piece is closed or open, wherein when the top end of the holding piece is closed, a cut is provided between the top end and a bottom end of the holding piece, the cut forms an angle in the range of 40 degrees to 50 degrees, and the thickness between a central portion of the cut and an inner wall of the holding piece is between 0.1 mm and 0.15 mm.

9. The bone fixation structure of claim 1, further comprising a connecting piece penetratingly disposed on the peripheral surface of the positioning member.

10. The bone fixation structure of claim 8, further comprising a fastening piece for fastening the connecting piece on the positioning member.

11. The bone fixation structure of claim 9, wherein the fastening piece is screwed inside the positioning member and presses against the connecting piece.

12. The bone fixation structure of claim 5, wherein the slot includes a narrow elongated slot, a fan-shaped slot or a slot having a combination of a circular portion and a narrow elongated portion, wherein the width of the narrow elongated slot is between 5.5 mm to 7.5 mm; the fan-shaped slot includes a wide portion between 7.0 mm to 9.5 mm and a narrow portion between 5.5 mm to 7.5 mm; and the slot having the combination of a circular portion and the narrow elongated portion includes a diameter of 7.0 mm to 9.5 mm for the circular portion and a width of 5.5 mm to 7.5 mm for the narrow elongated portion.

13. The bone fixation structure of claim 7, wherein the slot includes a narrow elongated slot, a fan-shaped slot or a slot having a combination of a circular portion and a narrow elongated portion, wherein the width of the narrow elongated slot is between 5.5 mm to 7.5 mm; the fan-shaped slot includes a wide portion between 7.0 mm to 9.5 mm and a narrow portion between 5.5 mm to 7.5 mm; and the slot having the combination of a circular portion and the narrow elongated portion includes a diameter of 7.0 mm to 9.5 mm for the circular portion and a width of 5.5 mm to 7.5 mm for the narrow elongated portion.
